(19) United States
(12) Patent Application Publication
(10) Pub. No.: US 2018/0289253 A1
(43) Pub. Date: Oct. 11, 2018

(54) METHODS AND SYSTEMS FOR PATIENT SPECIFIC IDENTIFICATION AND ASSESSMENTOF OCULAR DISEASE RISK FACTORS AND TREATMENT EFFICACY

(71) Applicant: INDIANA UNIVERSITY RESEARCH & TECHNOLOGY CORPORATION, Indianapolis, IN (US)

(72) Inventors: Alon Harris, Indianapolis, IN (US); Giovanna Guidoboni, Carmel, IN (US)

(21) Appl. No.: 15/575,730

(22) PCT Filed: May 20, 2016
(86) PCT No.: PCT/US2016/033521

§ 371 Date: Nov. 20, 2017

Related U.S. Application Data
(60) Provisional application No. 62/165,304, filed on May 22, 2015.

(51) Int. Cl. A61B 3/00; A61B 3/10; A61B 3/16; A61B 5/0205; A61B 3/12; A61B 5/03; A61B 5/00; A61B 3/14; G16H 15/00; G16H 50/50

(52) U.S. Cl. CPC A61B 3/0025; A61B 3/1005; A61B 3/16; A61B 5/0205; A61B 3/1241; A61B 5/021; A61B 5/4848; A61B 5/7275; A61B 3/14; G16H 15/00; G16H 50/50; A61B 5/031

(57) ABSTRACT

This disclosure provides systems and methods for patient-specific identification and assessment of ocular disease risk factors and efficacy of various treatments. The systems and methods can include mathematically modeling an expected normal patient-specific value of one or more clinically observable properties using a patient-specific mathematical model that can be calibrated with patient-specific data. The expected normal patient-specific value can be compared with a measured patient-specific value. A greater difference between the expected and measured patient-specific values can correlate to greater ocular vasculature abnormalities.

### CROSS-REFERENCE TO RELATED APPLICATIONS

This application is related to, claims priority to, and incorporates by reference herein for all purposes U.S. Provisional Patent Application No. 62/165,304, filed May 22, 2015.

### STATEMENT REGARDING FEDERALLY SPONSORED RESEARCH

This invention was made with government support under 1224195 awarded by the National Science Foundation. The Government has certain rights in the invention.

### BACKGROUND OF THE INVENTION

This invention relates to improved methods and systems for identifying and assessing ocular disease risk factors and testing efficacy of treatments.

Imaging of ocular tissues and description of their characteristics in health and disease remains both an area of great innovation and a current limitation in understanding ocular pathologies. Many ocular diseases such as glaucoma and various retinopathies have complex risk factor interactions that are not fully understood or well described in terms of individual risk factor assessment.

For example, many large clinical studies have shown that intraocular pressure, blood pressure, ocular perfusion pressure and ocular structure are important considerations in eye diseases such as glaucoma; however each of these are also often not associated with an individual's disease creating a contradiction in understanding disease risk. This is due to the fact that each variable individually is not capable of predicting disease; and each variable interacts with the other variables, with some interactions mitigating risk and other combinations resulting in elevated risk. For instance, an individual with a combination of intraocular pressure of X, blood pressure of Y, and ocular structure of Z may be at extreme risk for disease without one individual variable predicting this risk.

In another specific example, reduced ocular blood flow is associated with many ocular diseases and visual impairment. A question may then arise from clinicians: “When are changes in ocular blood flow critical? When we see reductions of 5%? 10% 15%?” This quantification of risk threshold cannot come from mere statistical analysis of clinical studies. There are just too many factors that interplay to determine ocular blood flow and these factors vary among individuals. In particular, ocular blood flow is driven by arterial blood pressure, impeded by intraocular pressure and modulated by vascular regulation. Using our innovative interdisciplinary method combining clinical data with mathematical modeling based on fluid-mechanics and mass-transport, we are now able to answer the crucial question raised by clinicians, namely “There is no absolute threshold that distinguish between critical and non-critical alteration in blood flow parameters, but this threshold must be individualized to the specific conditions of the patient”, and our mathematical model can quantitatively compute this individualized threshold. For example, our mathematical model can say that if a clinician measures a peak systolic velocity in the central retinal artery of 10 cm/s and an artero-venous difference in oxygen saturation of 30%, then these values are within normal range for a patient with intraocular pressure (TOP) equal to 15 mmHg and systolic and diastolic blood pressures (SBP/DBP) equal to 120/80 mmHg, but way too low for a patient with IOP=15 mmHg and SBP/DBP=140/90 mmHg.

Currently, there are only two algorithms available in ophthalmology: the Glaucoma 5-Year Risk Estimator (G5YRE); and the Pittsburgh Ocular Imaging Prefit Calculator (POIPC) for biomechanics predictions. Both have meaningful shortcomings.

The G5YRE is based on the data collected within the ocular hypertension study (OHTS) and the European Glaucoma Prevention Study (EGPS). The G5YRE has as inputs an individual patient's age, vertical cup/disc ratio by contour, intraocular pressure, central corneal thickness, pattern standard deviation and its only output is a percentage indicating the risk that the patient will develop primary open angle glaucoma within five years. The G5YRE is based on a large pool of clinical data acquired via the multicenter studies OHTS and EGPS. However, the calculator has limited clinical use due to the following limitations. First, there is no guarantee that the risk is accurate for an individual patient. Second, there is no indication of what might be the best therapeutic approach to prevent or delay the onset of glaucoma for a specific patient. Third, there is no indication regarding the potential risk of developing any other type of ocular disease or visual field loss. Finally, the calculator does not account for many other glaucoma risk factors, including ethnicity, myopia, and low perfusion pressure. The G5YRE is based on a large set of clinical data and sole multivariate statistical analysis does not allow the isolation of the role of each individual risk factor and does not explain how different factors interact to give the predicted level of risk for glaucoma development.

The POIPC is based on the modeling and experimental work performed within the laboratory of biomechanics at the University of Pittsburgh. The POIPC has as inputs a list of geometrical parameters of the sclera, lamina cribrosa and prelaminar tissue that are measurable or potentially measurable and as outputs strain, stresses, and material properties of the sclera, lamina cribrosa, and prelaminar tissue. However, the POIPC has no clinical use because the outputs are not measurable clinically. As a consequence, it is not possible to design a clinical study that can provide data on the relationship between alterations in stresses and strains and the development and progression of optic neuropathies. Moreover, POIPC accounts only for biomechanical factors (i.e., geometrical and material properties of ocular tissues) without considering their interplay with ocular blood flow through the tissue, which determines nutrient transport and delivery.

Accordingly, it would be beneficial to develop systems and methods that overcome the shortcomings of the existing algorithms and which are capable of patient-specific mathematically modeling of ocular disease risk factors, both clinical and theoretical. Such systems and methods could provide new diagnostic and treatment paradigms for ocular diseases and concrete tools for clinicians to better serve patients on an individualized basis. A need exists for systems and methods to achieve the aforementioned goals.

### SUMMARY OF THE INVENTION

The present invention overcomes the aforementioned drawbacks by providing systems and methods as described herein.

In accordance with one aspect of the present disclosure, a method of identifying ocular vasculature abnormalities in a patient can include one or more of the following steps: receiving, using a processor, patient-specific calibration data including age, height, and weight of the patient; calibrating, using the processor, a mathematical model using the patient-specific calibration data to generate a patient-specific mathematical model; receiving, using the processor, patient-specific input data including blood pressure, heart rate, intraocular pressure, and axial eye length of the patient; mathematically modeling, using the processor, an expected normal patient-specific value of one or more clinically observable properties, the mathematically modeling using the patient-specific input data and the patient-specific mathematical model or a non-specific mathematical model; and generating, using the processor, a report of the ocular vasculature abnormalities of a patient by comparing the expected normal patient-specific value of the one or more clinically observable properties with a measured patient-specific value of the one or more clinically observable properties, where a greater difference between the expected normal patient-specific value and the measured patient-specific value correlates to greater ocular vasculature abnormalities.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS AND EXHIBITS

FIG. 1 is a flowchart showing aspects of the methods of the present disclosure.

FIG. 2 is a flowchart showing aspects of the methods of the present disclosure.

FIG. 3 is a flowchart showing aspects of the methods of the present disclosure.

FIG. 4 is a schematic representation of the various blood vasculature used in one version of the mathematical model, in accordance with the present disclosure.

FIG. 5 is a schematic representation of components of one version of the mathematical model, in accordance with the present disclosure.

FIG. 6 is a plot showing clinically measured retinal arteriovenous difference in oxygen saturation for a healthy group and a glaucoma group, as described in Example 2.

FIG. 7 is a plot showing a norm value of a difference between a model predicted and clinically-measured values for a healthy group and a glaucoma group, as described in Example 2.

### DETAILED DESCRIPTION OF THE INVENTION

In General

As discussed briefly in the background section above, this disclosure represents a new approach to tackling the following questions: Is anything wrong in the eye of this specific patient that makes her a suspect for the development and/or progression of ocular and/or systemic diseases?—or—What would the outcome and the efficacy of a specific treatment be on a specific patient?

Generally speaking, it is known how to measure and model certain clinical parameters with respect to the eye of a patient. This disclosure provides a new application of these existing tools that will enable a clinician to better use measured and modeled clinical parameters in diagnosing and assessing patients. For example, it is known how to measure the intraocular pressure (IOP) in a patient and mathematical models exist for modeling IOP as well. However, knowing a patient's IOP in a vacuum provides very little diagnostic information, because there does not currently exist a tool that is capable of informing a clinician as to what the IOP should be in this patient if they have a healthy eye (or alternatively, what IOP implies an eye abnormality).

Similarly, it is known that various treatments and/or medications can impact certain clinical parameters with respect to the eye of a patient. For example, IOP-lowering medications are known to impact these clinical parameters with respect to the eye of a patient, but the effect is not the same for all patients. Again, knowing a patient's IOP in response to a treatment and/or medication in a vacuum provides little information to a clinician, because there does not current exist a tool that is capable of informing a clinician as to what the IOP change in response to the treatment and/or medication should be in the patient (or alternatively, what IOP implies poor treatment efficacy).

Currently available algorithms are only focused on one disease of the eye, namely glaucoma, while the systems and methods described herein are useful for many diseases of the eye, the body and the brain. Specifically, with the systems and methods disclosed herein, clinicians are provided concrete elements for the early diagnosis and monitoring of ocular diseases (examples: glaucoma, age-related macular degeneration), systemic diseases (examples: diabetes, hypertension) and neurodegenerative disorders (examples: Alzheimer's disease, Parkinson's disease, Lewy Body Disease). Moreover, currently available algorithms are either data-based (G5YRE) or model-based (POIPC), but they do not combine data and models. Data-based algorithms have the advantage of building on real clinical data, but dot not provide accurate patient-specific predictions. Model-based algorithms have the advantage of modulating inputs to patient-specific measurements, but do not provide clinical validation of predicted outputs. The systems and methods disclosed herein are based on the synergistic combination of clinical data and mathematical models, thereby allowing the input of patient-specific data and providing clinically-relevant outputs.

### Definitions

As used herein, an “expected normal patient-specific value” or a “virtual good value” refers to a modeled value of an ocular property of a patient that corresponds to the value that would be expected if the patient had healthy eyes with no abnormalities.

As used herein, a “clinically observable property” refers to a property of a patient that can be measured using clinical procedures. Examples of clinically observable properties include, but are not limited to, data extracted from retinal images, such as retinal vessel diameters, central retinal artery equivalent, and central retinal vein equivalent, ocular pulse amplitude, blood velocity in retrobulbar vessels, retinal hemodynamics, and retinal oximetry. It should be appreciated that as technology evolves, and more properties become clinically observable, the newly observable property shall be referred to as a clinically observable property.

### Methods of the Present Disclosure

The present disclosure provides a method of identifying ocular vasculature abnormalities in a patient. Some aspects of this disclosure are computer-based and require the use of a processor, while some aspects can be in the form of a reference chart where certain values for comparison can be looked up by a clinician.

In a basic sense, the methods include comparing a calculated value of one or more clinically observable property with a measured value of the one or more clinically observable property. The calculated value can be mathematically modeled based on patient-specific input data. The mathematical model can be calibrated using patient-specific calibration data.

The patient-specific input data can include a particular patient's blood pressure, heart rate, intraocular pressure, axial eye length, cerebrospinal fluid pressure, retinal nerve fiber layer thickness, macular thickness, optic disc dimensions, and the like.

The patient-specific calibration data can include information about a particular patient including age, height, weight, ethnicity, current medication information, medical history information, central corneal thickness, surgical status (i.e., did the patient have any surgery?), and the like.

The clinically observable properties can include retinal vessel diameter, central retinal artery equivalent, central retinal vein equivalent, amplitude of intraocular pressure oscillations, period of intraocular pressure oscillations, blood velocity in retrobulbar vessels, including peak systolic velocity, end diastolic velocity, resistive index, full time profile along a cardiac cycle, area under the curves, presence or absence of well-defined peaks, systolic slope, and diastolic slope, total retinal blood flow, blood velocity and flow in retinal arteries, blood velocity and flow in retinal capillaries, blood velocity and flow in retinal veins, oxygen saturation in retinal arteries, oxygen saturation in retinal veins, vascular and hemodynamic parameters (including, but not limited to, vessel diameters, vascular architecture, blood velocity, blood flow) in the retina, choroid, and optic nerve head (such as those obtained with Optical Coherence Tomography (OCT) techniques, including, but not limited to, swept source OCT, spectral OCT and OCT angiography), and the like.

The methods can also include providing inputs that are not used in the mathematical modeling, but which are provided to a clinician to assist in the medical evaluation. Examples of these inputs that are not used in models include visual field, visual acuity, mean deviation, pattern standard deviation, advanced glaucoma intervention study score, contrast sensitivity, and the like.

The methods can also include providing outputs of the model that are not clinically observable properties. These outputs can be of interest to a clinician or other researchers using the methods described herein.

In certain aspects, some outputs of the model or clinically observable properties can be provided in arbitrary units only. In those cases, the values are useful for comparison with previous calculations and measurements on the same patient at the same anatomical location, but may not be applicable broadly across varying patient populations or may not be normalizable across a larger patient population.

According to one aspect, the method can include the following steps: optionally receiving, using a processor, patient-specific calibration data including age, height, and weight of the patient; optionally calibrating, using the processor, a mathematical model using the patient-specific calibration data to generate a patient-specific mathematical model; receiving, using the processor, patient-specific input data including blood pressure, heart rate, intraocular pressure, and axial eye length of the patient; mathematically modeling, using the processor, an expected normal patient-specific value of one or more clinically observable properties, the mathematically modeling using the patient-specific input data and the patient-specific mathematical model or a non-specific mathematical model; and generating, using the processor, a report of the ocular vasculature abnormalities of a patient by comparing the expected normal patient-specific value of the one or more clinically observable properties with a measured patient-specific value of the one or more clinically observable properties, where a greater difference between the expected normal patient-specific value and the measured patient-specific value correlates to greater ocular vasculature abnormalities. In some aspects, the method can include measuring the measured patient-specific value of one or more clinically observable properties by processing a fundus image.

In certain aspects, the method can include using the mathematical model to generate a chart that displays an expected normal patient-specific value of one or more clinically observable properties for a particular patient-specific input data. A clinician could look up the expected normal patient specific value for the corresponding input data, and proceed with the comparison with measured values. In some aspects, the chart can also be generated for varying patient-specific calibration data.

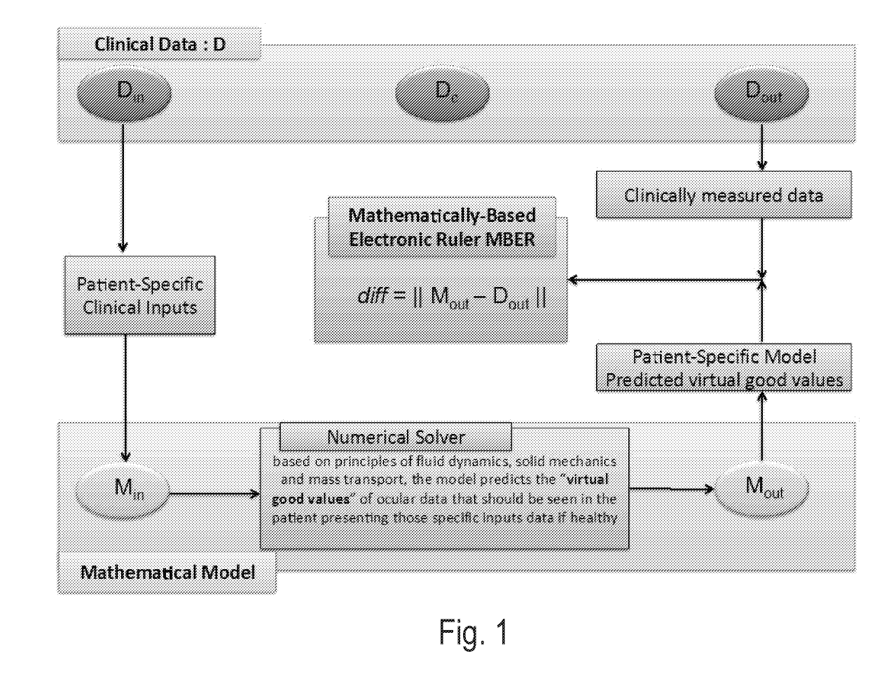

Referring to FIG. 1, a flowchart showing one aspect of the present disclosure is shown. The patient is evaluated at the clinic and a set of clinical data is collected. Let us denote this set by D. A software (it can be in the form of an app on phone or tablet, program on laptop, or other forms) based on a mathematical model of ocular biophysics (see below) is run with patient-specific inputs to provide simulated outputs of clinically measured quantities. We will denote model inputs and outputs as Mᵢₙ and Mₒᵤₜ. The model predicted outputs Mₒᵤₜ are the expected normal values for those quantities to be observed in an individual with the specific inputs Mᵢₙ. The set of measured clinical data D can be subdivided in three parts, namely D={Dᵢₙ,Dₒᵤₜ,D_{c}}. Dᵢₙ represents the clinical data that are used as patient-specific inputs in the mathematical model, so we set Mᵢₙ=Dᵢₙ. Dₒᵤₜ represents the clinical data that are compared with the outputs of the mathematical model, so we compute diff=∥Mₒᵤₜ−Dₒᵤₜ∥, where ∥⋅∥ indicates a suitable norm. Thus, the parameter diff is a measure of how much the clinical measurements Dₒᵤₜ differ from their expected patient-specific “virtual good value” Mₒᵤₜ predicted by the mathematical model with the patient-specific inputs Mᵢₙ. Thus, when diff is large the clinical condition of the patient noticeably deviates from its expected normal: for the clinician this means that something might be wrong with the patient making him a suspect for development and/or progression of ocular and/or systemic diseases. A further investigation of which quantities in the set Dₒᵤₜ (for example blood velocity in retinal or retrobulbar vessels, retinal oxygenation, aqueous humor flow, . . . ) are particularly off with respect to their mathematically computed “virtual good value” will provide clinicians concrete elements to devise the most effective personalized therapeutic approach or to estimate the outcomes of various therapeutic options. D, represents the clinical data that are not used as model inputs and that are not directly comparable with model outputs but are useful to the clinician for the patient evaluation.

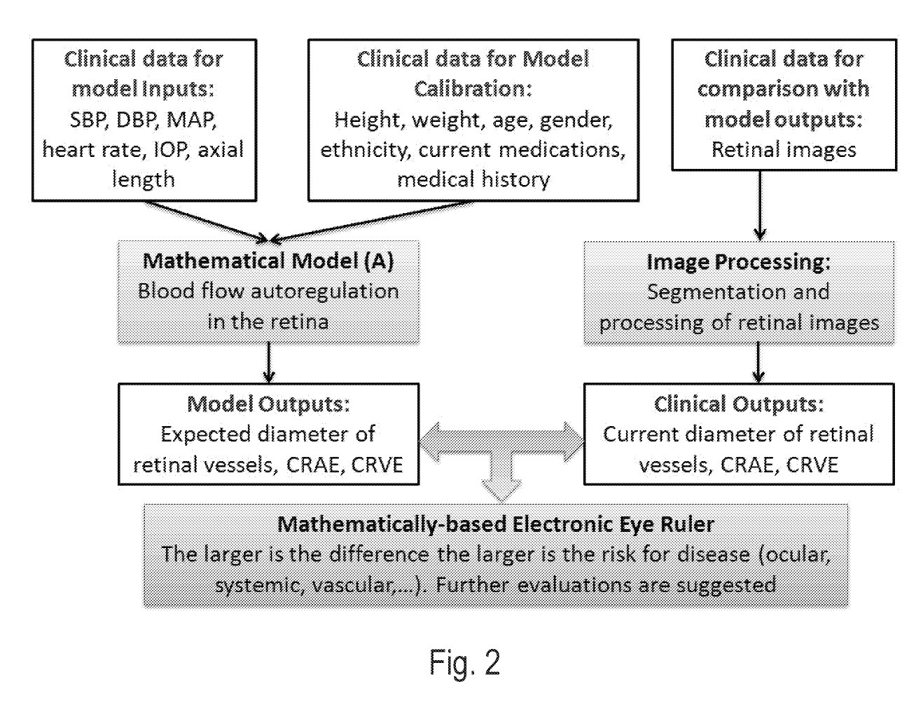

Referring to FIG. 2, a flowchart showing one aspect of the present disclosure is shown. The general operation is similar to that set forth above with respect to FIG. 1. The mathematical model deployed is described and the equations used can be found in Arciero et al., Invest Ophthalmol Vis Sci. 54(8) (2013) 5584-93. Briefly, blood is modeled as a Newtonian viscous fluid (with viscosity depending on the vessel size) flowing through an idealized retinal vasculature (see as an example the representative segment model in Arciero et al.). Oxygen and metabolites are transported in the blood stream and can diffuse in the tissue. Retinal venules passively deform under IOP. Retinal arterioles can alter their diameter as a consequence of changes in their wall tension. Wall tension has active and passive components. Active tension varies with an activation function which depends, in turns, on a stimulus function comprising various mechanisms, including (but not limited to) myogenic response, shear stress response, metabolic response, carbon dioxide (CO₂) response. Oxygen delivery is modeled via a Krogh-cylinder model and tissue metabolic demand is modeled via a Michaelis-Menten dynamics. The balance between tension, activation, metabolic demand and blood flow dictates the final diameter of retinal arterioles.

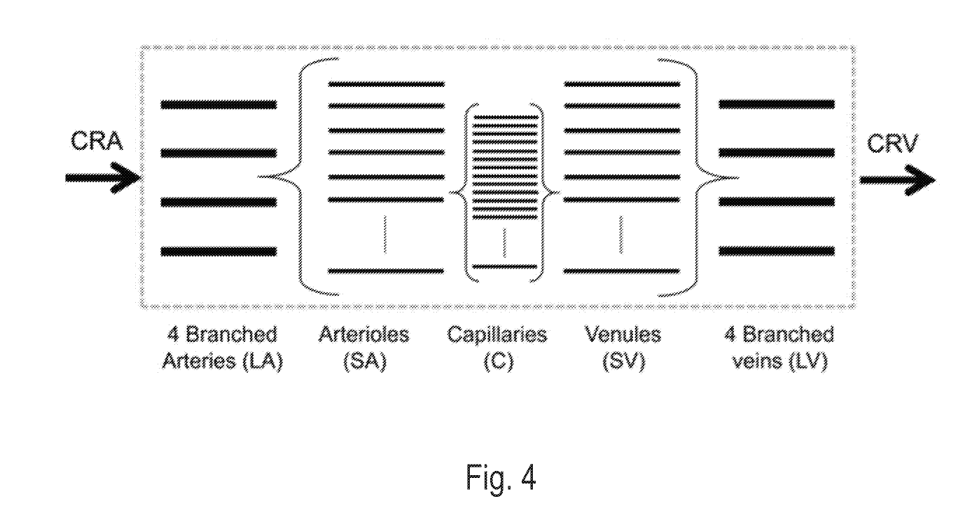

The model described briefly in the preceding paragraph will now be described in more detail. It should be appreciated that this is merely one exemplary model that can be used with the methods and systems described herein and that the present disclosure is not intended to be limited to any specific model. The retinal vasculature downstream of the central retinal artery (CRA) and upstream of the central retinal vein (CRV) is modeled as a representative segment network, where five vessel compartments for the large arterioles (LA), small arterioles (SA), capillaries (C), small venules (SV) and large venules (LV) supplying and draining the retina are connected in series; each compartment consists of identical segments arranged in parallel (see FIG. 4). All compartments are assumed to experience the same hemodynamic and metabolic conditions. A summary of the model equations, along with its input values and output values, is provided in Table 1. The subscripts used in Table 1 indicate if the quantity is evaluated either at the inlet (in,i), midpoint (mp,i) or outlet (out,i) of the i-th compartment, where i=LA,SA,C,SV,LV. The values of the model parameters are listed in Tables 2, 3 and 4. In the following, the main features of the model are described.

Blood flow and oxygen saturation throughout the network are predicted according to hemodynamic and mechanical principles. Retinal flow is assumed to follow Poiseuille's Law, in which flow through each vessel is proportional to the fourth power of the vessel diameter. The complex blood rheology is accounted for by assigning different values of the apparent viscosity μ to vessels in each compartment according to an experimental in vivo relationship (Table 4). The total tension T_{total,i} generated in the vessel walls of the vasoactive compartments i=LA, SA follows the Law of Laplace and is modeled as the sum of passive and active tension, denoted by T_{passive,i} and T_{max.active,i}, respectively, as detailed in Table 1(b). T_{passive,i} results from the structural components of the vessel wall, and T_{max.active,i} is generated by the contraction and dilation of smooth muscles in the LA and SA. Smooth muscle tone in LA and SA is described by the activation function A_{total,i}, which ranges from 0 to 1. The product of T_{max.active,i} and the activation A; yields the active tension generated in the vessel wall. Changes in A_{total,i} are dictated by the stimulus function S_{tone,i} which results from a linear combination of four autoregulatory mechanisms:

1) myogenic mechanism, related to the wall tension Tᵢ computed via the Law of Laplace. Details are provided in Table 1(c), where ΔPₜₒₜ represents the total pressure drop along the retinal network from the outlet of the CRA to the inlet of the CRV, and ΔPᵢ represents the pressure drop along each segment of the i-th compartment. Similarly, Qₜₒₜ represents the total blood flow along the network and Qᵢ represents the blood flow in each segment of the i-th compartment, and Rₜₒₜ represents the resistance to flow offered by the whole retinal network and Rᵢ represents the resistance to flow offered by a single segment of the i-th compartment. The resistances Rᵢ are computed according to Poiseuille's Law;

2) shear stress mechanism, related to the wall shear stress τᵢ computed according to Poiseuille's Law. Details are provided in Table 1(d);

3) metabolic mechanisms, related to the signal S_{CR,i}. Details are provided in Table 1(e), where the signal S_{CR,i} depends on the adenosine triphosphate (ATP) concentration at each position x along the network C(x), which itself depends on the blood oxygen saturation at each point in the network S(x). A Krogh cylinder model is used to predict oxygen diffusion in retinal tissue; here co is the oxygen carrying capacity of red blood cells at 100% saturation, H_{D} is the discharge hematocrit, q is the tissue oxygen consumption per vessel length and M₀ is the tissue oxygen demand per tissue volume (assumed here to be constant at 1.65 cm³ O₂·100 cm⁻³ min⁻¹). We remark that H_{D} could be set using patient-specific hematocrit values or, in the absence of such information, could be assumed to be constant, as specified in Table 3; and

4) carbon dioxide mechanism, related to the signal S_{CO2,LV}. Details are provided in Table 1(f), where the signal S_{CO2,LV} is given by the nonlinear function ƒ of the partial pressure of carbon dioxide in the tissue (PCO2) and of the total retinal blood flow (Qₜₒₜ). The tissue carbon dioxide content (tCO2) is converted into PCO2 via carbon dioxide dissociation curves, represented by the function g. The tissue carbon dioxide content and the blood carbon dioxide content (bCO2) are assumed to be linearly related.

In the model, autoregulation is achieved through changes in the diameters Dᵢ of the LA and SA segments, which should be interpreted as the new equilibrium state attained by the system as the input data are altered. Switching off metabolic and carbon dioxide mechanisms simulates the case of impaired autoregulation. Given the model inputs listed in Table 1 section (a), the steady-state values of the diameters Dᵢ and of the vascular smooth muscle activations A, in the LA and SA compartments are determined by integrating the system of ordinary differential equation in Table 1(b) until equilibrium is reached. It is important to note that the system also involves the quantities Tᵢ, T_{total,i} and A_{total,i} which, as detailed in Table 1, are functions of the unknowns Dᵢ and Aᵢ.

TABLE 1
Summary of Model Variables, Equations, Inputs, and Outputs.
Input

| | |
|---|---|
| (a) | P_{in,LA} = ⅔ MAP − 20 mmHg, P_{out,LV} = IOP, S(x = 0) = arterial oxygen saturation, |
| | M₀, d, bCO₂(x = 0) = 50%, C(x = 0) = 0.5 μM |

Equations

| (b) | $\{ \begin{matrix}\begin{matrix}\frac{{dD}_{i}}{dt} = \frac{2 {λ}_{i}}{{τ}_{d}} ( {T}_{i} - {T}_{total , i} ) \\ \frac{{dA}_{i}}{dt} = \frac{1}{{τ}_{a}} ( {A}_{total , i} - {A}_{i} )\end{matrix} & i = LA , SA\end{matrix}$ | |
|---|---|---|
| | T_{total,i} = T_{passive,i} + AᵢT_{max,active,i} T_{passive,i} = C_{pass,i} exp [C_{pass,i}′(Dᵢ/D_{0,i} − 1)] | |
| | T_{max,active,i} = C_{act,i} exp{−[(Dᵢ/D_{0,i} − C_{act,i}′)/C_{act,i}″]²} A_{total,i} = 1/(1 + exp(−S_{tone,i})) | |
| | S_{tone,i} = C_{myo,i}Tᵢ − C_{shear,iTi} − C_{meta,i}S_{CR,i} − C_{CO}_{₂}_{,i}S_{CO}_{₂}_{,LV} + C_{tone,i}″ | |
| (c) | Myogenic | Tᵢ = (P_{mp,i} − IOP)Dᵢ/2 i = LA, SA, C, SV, LV |
| | | ΔPₜₒₜ = QₜₒₜRₜₒₜ = P_{in,LA} − P_{out,LV} Rₜₒₜ = ΣᵢRᵢ Qₜₒₜ = nᵢQᵢ |
| | | ΔPᵢ = QᵢRᵢ = P_{in,i} − P_{out,i} Rᵢ = (128 μᵢLᵢ)/(πDᵢ⁴nᵢ) P_{mp,i} = P_{in,i} + ½ΔPᵢ |
| (d) | Shear stress | τᵢ = (32 μᵢQᵢ)/(πDᵢ³) i = LA, SA, C, SV, LV |
| (e) | Metabolic | $\begin{matrix} & {S}_{CR , i} = {\int }_{{x}_{mp , i}}^{{x}_{end , i}} exp [ - ( y - {x}_{mp , i} ) / {L}_{0} ] C ( y ) d y i = LA , SA \\ & C ( x ) = α + β ( x - {x}_{in , i} ) + exp [ γ ( {x}_{in , i} - x ) ] ( C ( {x}_{in , i} ) - α ) \\ & α ( x ) = {H}_{T} {R}_{0} [ {D}_{i} ( 1 - {R}_{1} S ( {x}_{in , i} ) ) - ( 1 - {H}_{D} ) {R}_{0} q ( x ) / ( π {c}_{0} {H}_{D} {k}_{d} ) ] / 4 {k}_{d} \\ & β ( x ) = ( {D}_{i} {H}_{T} {R}_{0} {R}_{1} q ( x ) ) / ( 4 {Q}_{i} {c}_{0} {H}_{D} {k}_{d} ) γ = {k}_{d} π {D}_{i} / [ ( 1 - {H}_{D} ) {Q}_{i} ] \\ & q ( x ) = {M}_{0} π ( {r}_{t , i}^{2} - {r}_{v , i}^{2} ) {r}_{v , i} = \frac{1}{2} {D}_{i} {r}_{t , i} = {r}_{v , i} + {d}_{i} \\ & S ( x ) = S ( {x}_{in , i} ) + q ( x ) ( {x}_{in , i} - x ) / ( {Q}_{i} {c}_{0} {H}_{D} ) \\ & {PO}_{2} ( x , r ) = {PO}_{2} ( x , {r}_{v , i} ) + {M}_{0} [ ( {r}^{2} - {r}_{v , i}^{2} ) / 4 + {r}_{t , i}^{2} ln ( {r}_{v , i} / r ) / 2 ] / k\end{matrix}$ |
| (f) | Carbon | S_{CO}_{₂}_{,LV} = f(PCO_{2LV},Qₜₒₜ) |
| | dioxide | PCO_{2LV} = g(tCO₂(x_{mp,LV})) tCO₂(x) = bCO₂(x)(1 − (−0.115Qₜₒₜ + 0.23)) |
| | | bCO₂(x) = bCO₂(x_{in,i}) − 0.81q(x)(x_{in,i} − x)/(Qᵢc₀H_{D}) |

Output

| | |
|---|---|
| (g) | Pᵢ, ΔPᵢ, Rᵢ and Qᵢ i = LA, SA, C, SV, LV, C(x), S(x), PO₂(r, x), tCO₂(x), bCO₂(x) |

TABLE 2
Parameter values for passive tension, active tension, and vascular smooth
muscle activation equations in the large arterioles (LA) and small arterioles (SA).

| | Value | | | | Value | | |
|---|---|---|---|---|---|---|---|
| Constant | LA | SA | Unit | Constant | LA | SA | Unit |
| Cₚₐₛₛ | 361.48 | 197.01 | [dyn/cm] | C_{Riya} | 0.0092 | 0.025 | [cm/dyn] |
| Cₚₐₛₛ′ | 53.69 | 17.60 | [1] | Cₛₕₑₐᵣ | 0.0258 | 0.0258 | [cm²/dyn] |
| C_{act} | 2114.2 | 3089.6 | [dyn/cm] | C_{cheta} | 200 | 200 | [μM/cm] |
| C_{act}′ | 0.93 | 1.02 | [1] | C_{co}₂ | 8e⁻⁴ | 1.31e⁻⁴ | [1/mmHg] |
| C_{act}^{φ} | 0.11 | 0.20 | [1] | Cₜₒₙₑ^{κ} | 159.26 | 62.27 | [1] |
| λ | 0.0457 | 0.0604 | [1/mmHg] | D₀ | 135.59 | 73.9 | [μm] |

TABLE 3
Time constants for the model equations (Table 1(b)) and
parameter values for the metabolic response (Table 1(e)).

| Parameter | Value | Unit |
|---|---|---|
| time constant for diameter, τ_{d} | 1 | [s] |
| time constant for activation, τₐ | 60 | [s] |
| tube hematocrit, Hₜ | 0.3 | [1] |
| discharge hematocrit, H_{D} | 0.4 | [1] |
| rate of ATP degradation, k_{d} | 2e⁻⁴ | [cm/s] |
| maximum rate of ATP release, Rₒ | 1.4e⁻⁹ | [mol s⁻¹ cm⁻³] |
| effect of oxygen saturation on ATP release, R₁ | 0.891 | [1] |
| oxygen capacity of red blood cells, C₀ | 0.5 | [cm³ O₂/cm³] |
| oxygen tissue diffusion coefficient, k | 9.4 | [cm³ O₂ cm⁻¹ |
| | | mmHg⁻¹ s⁻¹] |
| length constant for S_{cr}, L₀ | 1 | [cm] |

TABLE 4
Parameter values describing vessel network
geometry and viscosity.

| | Value | | | | | |
|---|---|---|---|---|---|---|
| Parameter | LA | SA | C | SV | LV | Unit |
| number of | 4 | 39 | 111,360 | 39 | 4 | [1] |
| segments, n | | | | | | |
| segment length, L | 0.807 | 0.583 | 0.088 | 0.583 | 0.807 | [cm] |
| viscosity, μ | 2.28 | 2.06 | 10.01 | 2.09 | 2.44 | [cP] |

As research in the modeling of the eye continues, the model for retinal blood flow autoregulation will become richer and more detailed. In addition to the features described in the preceding paragraphs, there are also optional features that can be included, alone or in combination, as follows. Blood is modeled as a non-Newtonian viscous fluid. Blood flow is simulated through a more realistic geometry, based, for example, on fractal trees reconstructions or on a patient-specific retinal vascular geometry reconstructed from retinal images. Central retinal artery and vein are also modeled along with the action of the IOP-induced compression of the lamina cribrosa on the vessel walls. The whole wall stress tensor (rather than merely the wall tension) is modeled via the three-dimensional theory of elasticity or viscoelasticity. The stimulus function can depend on other mechanisms, possibly dictated by local changes in ionic currents altering smooth muscle tone. Capillaries and other vascular segments (and not only arterioles) might be capable of actively change their diameter. Oxygen delivery to the tissue is modeled via the coupling between the oxygen transported in the superficial and deep capillary plexi and the layered structure of the retinal tissue. The model accounts for the time-pulsatility of blood flow.

The utilization of clinical data as model inputs (i.e., patient-specific input data) can vary based on the features of the particular model that is chosen. In the case of blood pressure and heart rate as model inputs, when using a model containing only basic features, the model is time independent, and therefore only steady state values of inputs/outputs are utilized and predicted. Mean arterial pressure (MAP=⅔ DBP+⅓ SBP) is used as driving force of the blood flow through the vasculature, whereas SBP, DBP might be used as inputs for a more advanced time-dependent version of the model. In the case of IOP as a model input, IOP is utilized as a given value for the external pressure acting on the intraocular vasculature. In the case of axial length of the eye as a model input, axial length is used to determine, via Laplace's law, the scleral tension acting on the lamina cribrosa for a given IOP value. This is an option in the additional features of the model, and therefore this clinical input will be utilized in advance option models and not in the model containing only basic features.

Patient-specific calibration data can be used to adjust some model parameters to increase the accuracy of model predictions. Information on height, weight, age, gender, ethnicity, current medications, medical history might be used to adjust some of the model parameters in order to increase the accuracy of model predictions. Such adjustment is not essential for the functioning of the methods, but does result in more accurate patient-specific results. Height, weight, age, gender, ethnicity, medications, ocular and/or systemic diseases might alter many model parameters, including but not limited to, total blood volume, which defines the reference hemodynamic state of the model, or mechanical properties of vascular walls, including but not limited to, rigidity, stiffness, or viscoelasticity, which define the passive response of blood vessels to trans-mural pressure differences.

Measured patient-specific values of clinically observable properties can be measured by analyzing images of ocular fundus. Images of ocular fundus can be obtained using various instruments, such as a fundus camera and optical coherence tomography. Several software tools are already available to segment images of the ocular fundus and extract relevant information about the diameter of retinal vessels. The diameter values retrieved with the software will be compared with model predictions and their difference will allow detecting and grading abnormal conditions.

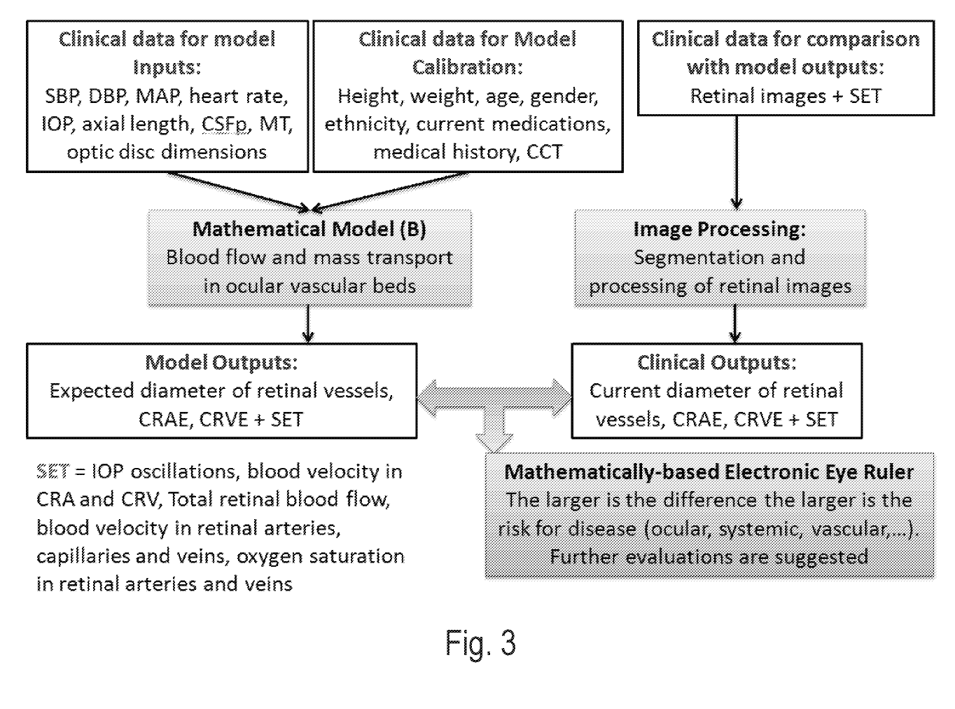

Referring to FIG. 3, a flowchart showing one aspect of the present disclosure is shown. The general operation is similar to that set forth above with respect to FIG. 1. The mathematical model in this aspect represents blood flow and mass transport in ocular vascular beds. Vascular beds of particular interests are those nourishing the retina, the choroid, the optic nerve head and the ciliary body. Modeling these various vascular beds has the following important implications. Most of the clinical measurements currently available pertain the retina. Vascular and functional alterations of the retina and the optic nerve head have been associated to various ocular and systemic diseases. Visualization of the optic nerve head vasculature and tissue in vivo in humans is still in its infancy. Ciliary body circulation is related to the build-up of IOP whose abnormal levels play an important role in glaucoma. The various vascular beds share the same arterial supply via the ophthalmic artery.

The various vascular beds can be modeled as coupled to one another. Different models can be used to model each vascular bed, or the same model can be used to model all vascular beds. Four different models for retinal circulation are discussed in the following references, which are incorporated herein in their entirety by reference: Arciero et al (2013) IOVS: Time-independent, without action of the IOP-compression of the lamina cribrosa on the central retinal vessels, retinal venules deformability model with Laplace's law, mechanistic description of blood flow autoregulation, idealized geometry for the vasculature, blood as a Newtonian fluid; Guidoboni et al (2014) IOVS: Time-dependent, with action of the IOP-compression of the lamina cribrosa on the central retinal vessels, retinal venules as Starling resistors, phenomelogical description of blood flow autoregulation, idealized geometry, blood as a Newtonian fluid; Cassani et al (2014) In P. Causin & G. Guidoboni & R. Sacco & A. Harris (Eds), Integrated Multidisciplinary Approaches in the Study and Care of the Human Eye (pp. 29-36). Amsterdam, the Netherlands: Kugler Publications: Time-independent, with action of the IOP-compression of the lamina cribrosa on the central retinal vessels, retinal venules deformability model with Laplace's law, mechanistic description of blood flow autoregulation, idealized geometry, blood as a Newtonian fluid; and P. Causin et al (2014). In P. Causin & G. Guidoboni & R. Sacco & A. Harris (Eds), Integrated Multidisciplinary Approaches in the Study and Care of the Human Eye (pp. 161-175). Amsterdam, the Netherlands: Kugler Publications: Time-independent, without action of the IOP-compression of the lamina cribrosa on the central retinal vessels, retinal vessels of fixed diameter, realistic geometry, blood as a non Newtonian fluid. For the circulation in the lamina cribrosa within the optic nerve head a poroelastic model has been developed (Causin et al (2014) Mathematical Biosciences). For the circulation in the choroid and ciliary body simplified electric circuits can be effective models (Guidoboni et al (2015) Annual Meeting of ARVO).

In order to account for realistic vascular geometries in the retina obtained via clinical imaging (e.g. via fundus camera or ocular coherence tomography (OCT) or OCT angio—but not limited to these), the circulation in the retina could be modeled as a network of one-dimensional vessels, or as a porous medium or as a hierarchical porous medium. Other vascular beds might also be included in the model. For example, the circulation in the ciliary body, in the choroid and in the optic nerve head, including the lamina cribrosa, can be modeled via simplified electric circuits or via poroelastic models.

In addition to the model inputs described elsewhere, additional inputs include the following: cerebrospinal fluid pressure, utilized to estimate the retrolaminar tissue pressure acting on the posterior surface of the lamina cribrosa; optic disc dimensions, utilized to estimate the dimensions of the lamina cribrosa; and macular thickness, utilized to set the thickness of the tissue layer at the macula, in the model versions accounting for realistic geometries of the retina.

In addition to the model calibration data described elsewhere, central corneal thickness can be utilized in the model for aqueous humor production/draining to set the geometrical properties for the cornea. The central corneal thickness can also be utilized in setting the geometric properties of the sclera and lamina cribrosa.

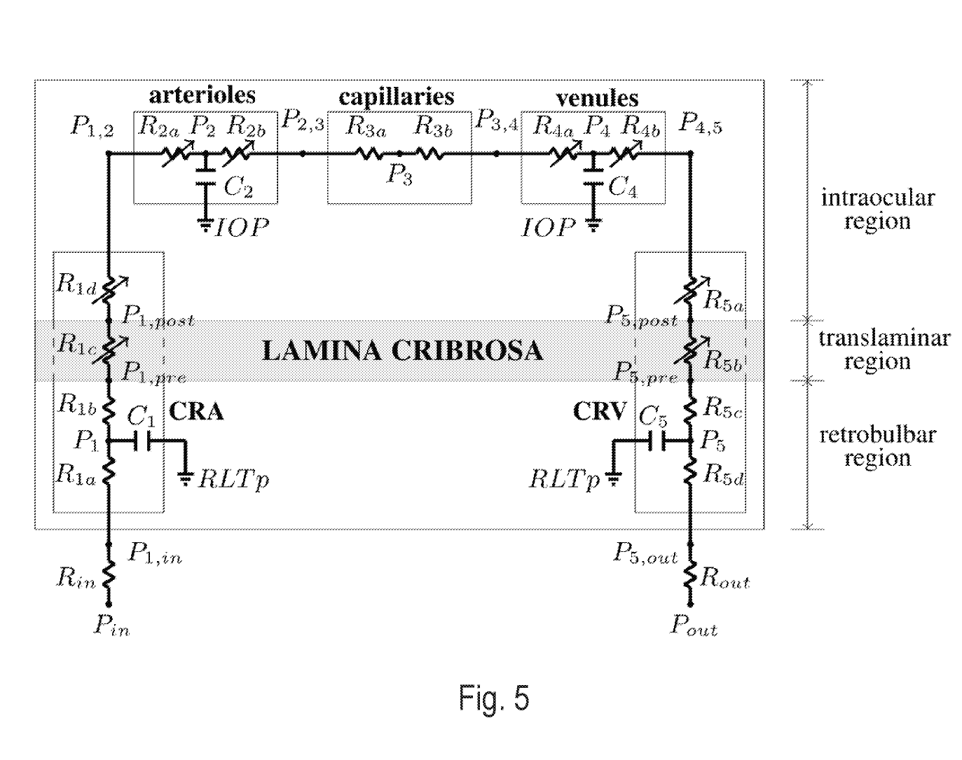

Referring to FIG. 5, a schematic representation of one example of a mathematical model suitable for use in the methods described herein is shown. For this mathematical model, the patient inputs include systolic and diastolic blood pressure, heart rate, intraocular pressure, central cornea thickness, and dimensions of the optic disc. This mathematical model is based on principles of fluid dynamics, solid mechanics and mass transport, and is used to simulate the blood flow and oxygen transport in the three vascular beds nourishing the retina, the choroid and the optic nerve head, and in the retrobulbar vessels (ophthalmic artery, central retinal artery and vein, nasal and temporal posterior ciliary arteries). Model inputs include patient-specific values of the quantities listed above, as well as literature-based values for the remaining geometrical, mechanical and physical properties. A schematic representation of the connection between the various vascular beds is provided in FIG. 5.

The retinal vasculature is represented by the model depicted in the FIG. 5. The vasculature is divided into five main compartments: the central retinal artery (CRA), arterioles, capillaries, venules, and the central retinal vein (CRV). Using the analogy between hydraulic and electrical circuits, blood flow is modeled as current flowing through a network of resistors (R), representing the resistance to flow offered by blood vessels, and capacitors (C), representing the ability of blood vessels to deform and store blood volume. The vascular segments are exposed to various external pressures depending on their position in the network. The intraocular segments are exposed to the IOP, the retrobulbar segments are exposed to the retrolaminar tissue pressure (RLTp), and the trans-laminar segments are exposed to an external pressure that depends on the internal state of stress within the lamina cribrosa. The IOP-induced stress within the lamina cribrosa is computed using a nonlinear elastic model. Arrows have been used in FIG. 5 to indicate all resistances that can vary, either passively or actively. The inlet and outlet pressures Pᵢₙ and Pₒᵤₜ vary with time along a cardiac cycle and, consequently, pressures, flows and velocities calculated by the model are time dependent.

The methods require only that “some” model for vascular beds is present and coupled with the others, but which specific model to use might differ depending on the needs. For example, if the clinic can perform Color Doppler Imaging (CDI) to measure the blood velocity in the retrobulbar vessels, then a time-dependent model for the retinal and retrobulbar circulation must be used, in order to compare clinical data and model predictions of peak systolic velocity and end diastolic velocity, see Guidoboni et al., Invest Ophthalmol Vis Sci. 55(7) (2014) 4105-18. This model is described in the paragraphs immediately preceding this paragraph.

If the clinic does not have the capability of performing CDI but is equipped with a Heidelberg Retinal Flowmeter (HRF) to measure blood flow, volume and velocity in the capillaries, a stationary model for the retinal circulation will suffice, see Arciero et al., Invest Ophthalmol Vis Sci. 54(8) (2013) 5584-93. This model is described above with respect to Tables 1-4.

Furthermore, depending on whether or not the clinic can perform retinal oximetry, mass transport in the retina may or may not be simulated. Depending on the computing capabilities of the clinic, perfusion in the lamina cribrosa may or may not be calculated. Current imaging techniques do not allow to visualize and measure blood flow parameters in the tissues of the optic nerve head, which include the lamina cribrosa, but these parameters might be important factors in many optic neuropathies (thus this feature is important for clinical research applications). As mathematical models are further developed, various options can be provided to implement in each particular software segment depending on the need of the specific user (for example clinic, research center, pharmaceutical and biotech company). Also, other segments might be added in the future.

Some model outputs are comparable to clinically measurable values and some are not currently measurable, but may be in the future. Examples of model outputs that can be compared with patient-specific data include the following: blood velocity and vascular resistance in the ophthalmic artery, central retinal artery and vein, and nasal and temporal posterial ciliary arteries can be compared to values measured in Color Doppler Imaging; total retinal blood volume can be compared to values measured via Fourier-Domain-Optical Coherence Tomography; blood flow, volume, and velocity in capillaries can be compared to values measured via a Heidelberg Retinal Flowmeter; and oxygen saturation in retinal arterioles and venules can be compared to values measured via retinal oximetry. Examples of model outputs having clinical relevance, but not currently comparable with measured data include intravascular pressure, blood flow, and blood velocity in all retinal vascular compartments, and pressure, blood flow, blood velocity, and diameter of blood vessels in all vascular compartments.

Model outputs and clinical data are compared for an individual patient. Differences between model outputs and clinical data provide a quantitative measure of the presence and severity of vascular abnormalities in that patient (larger differences indicate more severe abnormalities). The comparison between model outputs and clinical data can also be performed on a subset of the quantities listed in Point 3a depending on data availability. Model outputs that are not currently comparable with clinical data due to the lack of technology in humans still provide insightful information on the vascular health of the individual patient.

This disclosure also provides methods of predicting the effect, outcome, and/or efficacy of a given treatment, such as treatment with a medication or a specific procedure. These methods can involve executing the other methods described above with various changes to the inputs that accounts for the effect of the medication. For example, if a given medication is known to lower a patient's blood pressure, the effect of that medication can be modeled by using pre-treatment patient-specific input data (such as IOP level, systolic blood pressure, diastolic blood pressure, functionality of vascular regulation, age, and the like), estimating the effects of the medication (such as lowering blood pressure), and using values after accounting for the estimated effects as inputs in the models described herein. If the goal of administering a given medication is changing one or more of the outputs of the model described herein (such as IOP), the methods can provide insight into the effect of that medication without requiring administering the medication to the patient. As discussed elsewhere herein, while it might generally be known how various changes effected by a treatment can impact ocular properties, the methods described herein can provide patient-specific insight into the expected impact of the treatment to the ocular vasculature of a specific patient.

This disclosure further provides methods of evaluating the effect, outcome, and/or efficacy of a given treatment. These methods can involve acquiring patient-specific input data pre- and post-treatment and executing the methods described herein to provide model outputs for both sets of input data. These methods can also involve comparing outputs that are based on the predicted impact of the treatment with outputs that are based on the actual impact of the treatment using post-treatment input data.

It should be appreciated that, while the present disclosure is described in the greatest detail with respect to specific disease states, the present disclosure is not intended to be limited to those specific disease states and is applicable to more than one disease state (for example, glaucoma, age-related macular degeneration, diabetic retinopathy, stroke, hypertension, etc.). Many disease states share different risk factors and to the extent that the present disclosure is related to the identification of these personalized risk factors, the present disclosure is applicable to disease states that are related to those risk factors. Similarly, while the present disclosure is described in the greatest detail with respect to certain risk factors, the present disclosure is not intended to be limited to those specific risk factors and is applicable to more than one risk factor (for example, sleep apnea, blood pressure, retinal oxygenation, blood return, etc.). Many risk factors are applicable to different disease states and to the extent that the present disclosure is related to the identification and assessment of these personalized disease states, the present disclosure is applicable to risk factors that are related to those disease states.

Systems of the Present Disclosure

The present disclosure provides systems for identifying ocular vasculature abnormalities or testing various treatments in a patient. The systems can include a non-transitory, computer-readable memory having stored thereon a program for executing the methods described herein. The memory can also store patient data, as described herein.

The systems can also include a user interface for entering patient data.

In certain aspects, the systems can include a software running on a computer, or an app running on a tablet, smartphone, or other personal device. In certain aspects, the systems can include reference tables.

Clinical Applications

The present disclosure provides clinical applications of the systems and methods described herein.

Different risk factors can combine in unique ways to each individual to determine that individual's overall risk to develop a certain disease or to experience disease progression. The overall risk level is a major determinant in the choice of preventive therapies and clinical management. Clinicians have historically not had quantitative tools to aid in the determination of a patient's individual risk for disease and thus clinicians are left to rely on their experience and judgment. The following clinical applications describe how the systems and methods disclosed herein can improve clinical outcomes.

Non-artheritic ischemic optic neuropathy (NAION) is a disease of the optic nerve which entails ischemic damage and acute vision loss. Patients suffering from NAION experience immediate vision loss, either in one of both eyes. The vision loss can be total or limited to one hemisphere (altitudinal defect). Patients usually rush to the hospital and are administered blood thinners and/or are exposed to a barometric chamber. Earlier establishment of the risk of NAION can afford treatment with blood thinners earlier and the crisis might be averted. The systems and methods described herein can be used to estimate the risk for NAION. One of the key risk factors for NAION is related to the interplay between IOP and blood pressure. Ischemic conditions of the optic nerve head mainly occur when the blood pressure drops and/or IOP increases and the vasculature is not able to properly autoregulate to maintain the necessary blood flow and oxygen supply to the tissue. Such conditions might arise as a consequence of the circadian rhythm, where blood and intraocular pressures change between day and night, or of medications, such as VIAGRA®. Examples of the various medications include, but are not limited to, the following: medications altering blood pressure, such as VIAGRA® and anti-hypertensives, which might put individuals with vascular autoregulation problems at risk for vision loss; medications altering IOP, such as steroids, which might put individuals with vascular autoregulation problems at risk for vision loss; and any system medications or topical medications that may affect circulation.

The systems and methods described herein can be used to detect and grade vascular abnormalities in the eyes of each patient (as explained in the previous sections), thereby providing a quantitative estimate for the associated risk of developing NAION. Should the risk be high, the clinician has evidence on which to base decisions of preventive treatment for that specific patient.

Central retinal vein occlusion (CRVO) is similar to NAION. The risk for CRVO is related to the interplay between IOP and blood pressure. Thus the systems and methods disclosed herein can be used in a similar fashion.

Complications resulting from diabetes, such as heart attacks, strokes, and renal failures, are among the major causes of morbidity and mortality in diabetic patients. Despite this, there is a scarcity of noninvasive biomarkers for the early detection of these complications. The systems and methods described herein combine the clinically available information on the geometry of blood vessels, blood pressure, and IOP, and can provide a quantitative estimate of the risk for onset of diabetic complications in the eye by evaluating the status of the microvasculature. In addition, the systems and methods described herein can test efficacy and outcomes of various treatments (for example, IOP-lowering medications or trabeculectomy).

It should be appreciated that these clinical applications are exemplary, and that the systems and methods described herein can be used to detect and grade vascular abnormalities in the macro- and micro-circulation of patients at risk for many diseases, including but not limited to, glaucoma, diabetes, and hypertension.

### Example 1. Guiding Clinicians in Devising Therapeutic Approaches

Measurements from 89 healthy individuals and 74 glaucoma patients of age 40 years or older were collected. Individuals with systemic diseases or ocular diseases other than open angle glaucoma were excluded from the study. However, patients receiving antihypertensive medication for elevated systemic blood pressure and patients with mild cataracts were not excluded. Glaucoma was defined based on the characteristic optic disc damage and the corresponding visual field defects. Of all of the glaucoma patients considered in this study, 45 were diagnosed with primary open-angle glaucoma (POAG) and 29 were diagnosed with normal-tension glaucoma (NTG). A diagnosis of POAG was defined by an untreated IOP>21 mmHg. Patients with IOP measurements consistently 21 mmHg were classified as having NTG. All glaucoma patients underwent automated perimetry. A patient was defined as having “mild glaucoma” if the visual field mean defect (MD) was ≤5 dB and was defined as having “advanced glaucoma” if the visual field MD≥10 dB. Of the 45 POAG patients, 20 were diagnosed with mild glaucoma and 12 were diagnosed with advanced glaucoma. Of the 29 NTG patients, 13 were diagnosed with mild glaucoma and 9 were diagnosed with advanced glaucoma. Some patients were under active treatment at the time of measurement.

When applying only statistical analysis to the oximetry data in the clinical dataset, both the advanced POAG and advanced NTG patient groups exhibited a higher average value of venous oxygen saturation than healthy individuals, and a lower average value of arteriovenous difference than healthy individuals. No statistical difference was reported in retinal oxygen saturation when mild POAG and mild NTG patients were compared, nor when advanced POAG and advanced NTG patients were compared.

Model optimizations were run as follows: given clinical measurements of intraocular pressure (IOP), mean arterial pressure (MAP), and arterial oxygen saturation for each individual in the dataset, the model was used to predict the level of oxygen demand (M₀) that would yield the clinically-measured value of venous oxygen saturation in each population. Simulations were run with functional and impaired autoregulation.

The patient-specific model optimizations suggest that there might be different explanations for the increased venous saturation levels observed among advanced POAG patients and advanced NTG patients. Specifically, a decrease in oxygen demand may be more relevant to the increase in venous saturation observed in advanced POAG, while impaired autoregulation mechanisms may be more relevant to the increase in venous saturation observed in advanced NTG.

This example illustrates that vascular changes might occur primary to glaucomatous damage in NTG patients and therefore point to therapies where IOP and vascular parameters are jointly addressed in NTG patients.

### Example 2. Identifying and Assessing Abnormalities

The patient group from Example 1 was used in this Example.

Patient-specific simulations were run by setting MAP and IOP equal to their measured values in a specific patient, and by setting the values of the other inputs listed in Table 1 at baseline. In this case, we consider the baseline values of the metabolic consumption were set at M₀=1 cm³ O₂*100 cm⁻³ min⁻¹ and arterial oxygen saturation=0.9742. It should be appreciated that baseline values can be further tuned using larger datasets and/or accounting for the patient's age, gender, disease status, and other factors discussed elsewhere herein. The model was used to compute the arteriovenous difference in oxygen saturation to be expected in each patient. Then, the model predicted values of arteriovenous difference in oxygen saturation are compared with the values measured for each patient via retinal oximetry. Results are reported in the Table below for a dataset including 15 glaucoma patients and 15 age-matched healthy individuals. Three indices for the norm of the difference between model-predicted and clinically-measured values are defined here as follows:

diff_1=(clinically measured−model predicted)/clinically measured;

diff_2=(clinically measured−model predicted)*100/clinically measured; and

diff_3=clinically measured−model predicted.

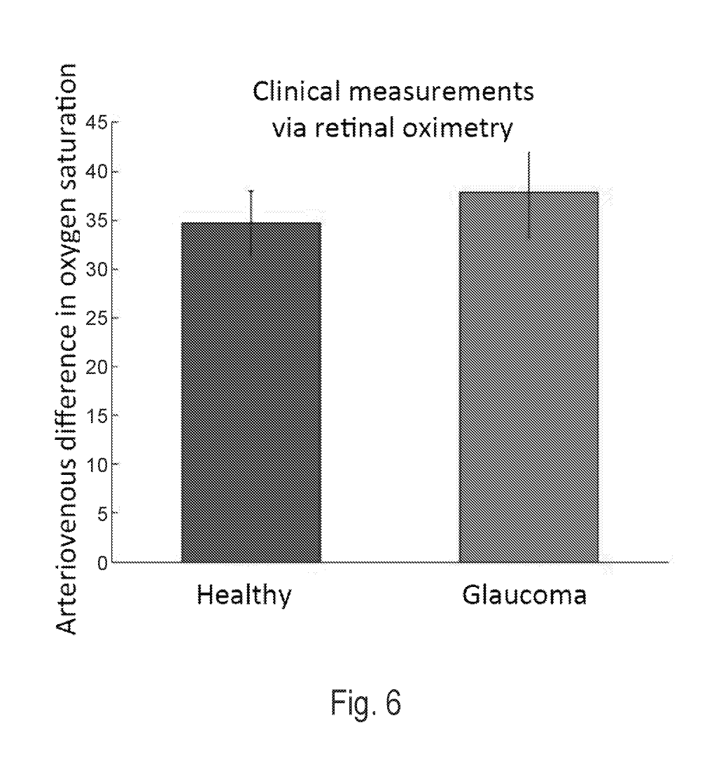

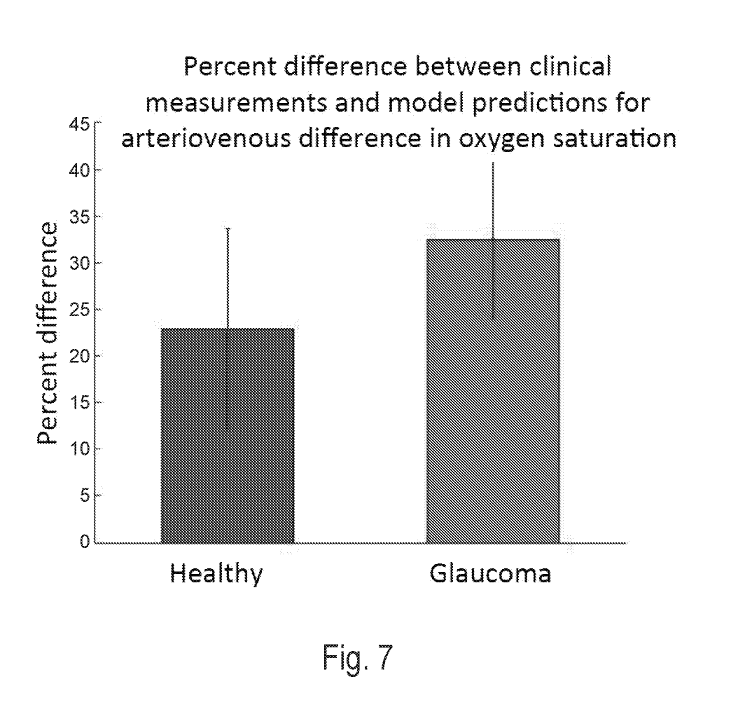

The results of the simulation are set forth in Table 5 and illustrated in FIGS. 6 and 7. FIG. 6 is a plot showing the clinically measured retinal arteriovenous difference in oxygen saturation for the healthy group and the glaucoma group. FIG. 7 is a plot showing the diff_2 norm for the healthy group and the glaucoma group. The results show that larger values of the calculated norms (diff_1, diff_2, and diff_3) correspond to the glaucoma group.

TABLE 5
Results of Simulation.

| | Retinal arteriovenous difference in oxygen saturation | | | | |
|---|---|---|---|---|---|
| Patient | Clinically | Model | | | |
| group | Measured | Predicted | diff_1 | diff_2 | diff_3 |
| Healthy | 34.7 ± 3.3 | 26.5 ± 2.3 | 0.23 ± 0.10 | 22.9 ± 10.8 | 8.2 ± 4.4 |
| Glau- | 37.8 ± 4.5 | 25.5 ± 2.0 | 0.32 ± 0.08 | 32.5 ± 8.6 | 12.8 ± 4.4 |
| coma | | | | | |

It should be appreciated that the definition of the norm can be defined by a clinical user. diff_1, diff_2, and diff_3 are all examples of norms and other possible norms can be utilized, depending on the clinical context.

### Prophetic Example 3. Outcomes of Medications and/or Treatments

Prolonged arterial hypertension leads to a thickening of the arterial walls; arterial wall thickness and elastic properties are parameters that can be incorporated in the methods described herein and can be changed to account for disease status. Systemic anti-hypertensive medications will lower the blood pressure, but their hemodynamic impact may vary among patients depending on how long they had hypertension before treatment and other health conditions. The methods described herein can be used to predict the hemodynamic outcome in a specific patient, as well as monitoring the patient status. The methods described herein can quantify additional outcomes in terms of drug-induced hemodynamic alterations in the ocular vascular beds.

Obviously, many modifications and variations of the present invention are possible in light of the above teachings and may be practiced otherwise than as specifically described while within the scope of the appended claims.

The following list of references are incorporated herein in their entirety by reference:
G. Guidoboni, A. Harris, S. Cassani, J. Arciero, B. Siesky, A. Amireskandari, L. Tobe, P. Egan, I. Januleviciene, J. Park, Intraocular Pressure, blood pressure and retinal blood flow autoregulation: a mathematical model to clarify their relationship and clinical relevance. Invest Ophthalmol Vis Sci. 55(7) (2014) 4105-18.
J. Arciero, A. Harris, B. Siesky, A. Amireskandari, V. Gershuny, A. Pickrell, G. Guidoboni, Theoretical analysis of vascular regulatory mechanisms contributing to retinal blood flow autoregulation. Invest Ophthalmol Vis Sci. 54(8) (2013) 5584-93.
G. Guidoboni, A. Harris, L. Carichino, Y. Arieli, B. A. Siesky, Effect of Intraocular Pressure on the Hemodynamics of the Central Retinal Artery: a Mathematical Model. Math Biosci Eng. 11(3) (2014) 523-46.
P. Causin, G. Guidoboni, A. Harris, D. Prada, R. Sacco, S. Terragni, A poroelastic model for the perfusion of the lamina cribrosa in the optic nerve head. Math Biosci. 257 (2014) 33-41.
P. Causin et al (2014). In P. Causin & G. Guidoboni & R. Sacco & A. Harris (Eds), Integrated Multidisciplinary Approaches in the Study and Care of the Human Eye (pp. 161-175). Amsterdam, the Netherlands: Kugler Publications.
S. Cassani, G. Guidoboni, I. Janulviciene, L. Carichino, B. A. Siesky, L. A. Tobe, A. Amireskandari, D. P. Baikstiene, A. Harris, Effect of trabeculectomy on retinal hemodynamics: mathematical modeling of clinical data (2014). In P. Causin, G. Guidoboni, R. Sacco and A. Harris (Eds), Integrated Multidisciplinary Approaches in the Study and Care of the Human Eye (pp. 29-36). Amsterdam, the Netherlands: Kugler Publications.
P. Causin, G. Guidoboni, F. Malgaroli, R. Sacco, A. Harris, Blood flow mechanics and oxygen transport and delivery in the retinal microcirculation: multiscale mathematical modeling and numerical simulation. Biomech Model Mechanobiol. 2016 June; 15(3): 525-42 [Epub 2015 Aug. 1].
L. Carichino, A. Harris, G. Guidoboni, B. A. Siesky, E. Vandewalle, O. B. Olafsdottir, S. H. Hardarson, K. van Keer, I. Stalmans, E. Stefánsson, J. C. Arciero, A theoretical investigation of the increase in venous oxygen saturation levels in advanced glaucoma patients. Journal for Modeling in Ophthalmology 1(1) (2016) 64-87.
A. Dziubek, G. Guidoboni, A. Harris, A. H. Hirani, E. Rusjan, W. Thistleton. Effect of ocular shape and vascular geometry on retinal hemodynamics: a computational model. Biomechanics and Modeling in Mechanobiology. 2015 Oct. 7 [Epub ahead of print].
M. Szopos, S. Cassani, G. Guidoboni, C. Prud'homme, R. Sacco, B. Siesky, A. Harris, Mathematical modeling of aqueous humor flow and intraocular pressure under uncertainty: towards individualized glaucoma management. Journal for Modeling in Ophthalmology. Accepted (April 2016).
A. G. Mauri, L. Sala, P. Airoldi, G. Novielli, R. Sacco, S. Cassani, G. Guidoboni, B. A. Siesky, A. Harris, Electro- fluid dynamics of aqueous humor production: simulations and new directions. Journal for Modeling in Ophthalmology. Submitted (February 2016).

The present invention has been described in terms of one or more preferred embodiments, and it should be appreciated that many equivalents, alternatives, variations, and modifications, aside from those expressly stated, are possible and within the scope of the invention.

1. A method of identifying ocular vasculature abnormalities in a patient, the method comprising:
a) optionally receiving, using a processor, patient-specific calibration data including age, height, and/or weight of the patient;
b) optionally calibrating, using the processor, a mathematical model using the patient-specific calibration data to generate a patient-specific mathematical model;
c) receiving, using the processor, patient-specific input data including blood pressure, heart rate, intraocular pressure, and/or axial eye length of the patient;
d) mathematically modeling, using the processor, an expected normal patient-specific value of one or more clinically observable properties, the mathematically modeling using the patient-specific input data and the patient-specific mathematical model or a non-specific mathematical model; and
e) generating, using the processor, a report of the ocular vasculature abnormalities of a patient by comparing the expected normal patient-specific value of the one or more clinically observable properties with a measured patient-specific value of the one or more clinically observable properties, where a greater difference between the expected normal patient-specific value and the measured patient-specific value correlates to greater ocular vasculature abnormalities.

2. The method of claim 1, the patient-specific calibration data including ethnicity, current medication information, and medical history information of the patient.

3. The method of claim 1, the patient-specific calibration data including central corneal thickness.

4. The method of claim 1, the patient-specific input data including cerebrospinal fluid pressure, retinal nerve fiber layer thickness, macular thickness, or optic disc dimensions.

5. The method of claim 1, wherein the one or more clinically observable properties include retinal vessel diameter, central retinal artery equivalent, central retinal vein equivalent, or a combination thereof.

6. The method of claim 5, the method further comprising:
e0) measuring the measured patient specific value of the one or more clinically observable properties by processing a fundus image.

7. The method of claim 5, wherein the one or more clinically observable properties include amplitude of intraocular pressure oscillations, period of intraocular pressure oscillations, blood velocity in retrobulbar vessels, total retinal blood flow, blood velocity in retinal arteries, blood velocity in retinal capillaries, blood velocity in retinal veins, oxygen saturation in retinal arteries, oxygen saturation in retinal veins, or a combination thereof.

8. The method of claim 7, wherein the blood velocity in retrobulbar vessels includes one or more of the following: peak systolic velocity, end diastolic velocity, resistive index, full time profile along a cardiac cycle, area under the curves, presence or absence of well-defined peaks, systolic slope, and diastolic slope.

9. A system comprising a processor and a non-transitory, computer-readable memory having stored thereon instructions that, when executed by the processor, cause the processor to:
a) optionally receive, using the processor, patient-specific calibration data including age, height, and/or weight of the patient;
b) optionally calibrate, using the processor, a mathematical model using the patient-specific calibration data to generate a patient-specific mathematical model;
c) receive, using the processor, patient-specific input data including blood pressure, heart rate, intraocular pressure, and/or axial eye length of the patient;
d) mathematically model, using the processor, an expected normal patient-specific value of one or more clinically observable properties, the mathematically modeling using the patient-specific input data and the patient-specific mathematical model or a non-specific mathematical model; and
e) generate, using the processor, a report of the ocular vasculature abnormalities of a patient by comparing the expected normal patient-specific value of the one or more clinically observable properties with a measured patient-specific value of the one or more clinically observable properties, where a greater difference between the expected normal patient-specific value and the measured patient-specific value correlates to greater ocular vasculature abnormalities.

10. The system of claim 9, the patient-specific calibration data including ethnicity, current medication information, and medical history information of the patient.

11. The system of claim 9, the patient-specific calibration data including central corneal thickness.

12. The system of claim 9, the patient-specific input data including cerebrospinal fluid pressure, retinal nerve fiber layer thickness, macular thickness, or optic disc dimensions.

13. The system of claim 9, wherein the one or more clinically observable properties include retinal vessel diameter, central retinal artery equivalent, central retinal vein equivalent, or a combination thereof.

14. The system of claim 13, the instructions, when executed by the processor, further cause the processor to:
e0) measure the measured patient specific value of the one or more clinically observable properties by processing a fundus image.

15. The system of claim 13, wherein the one or more clinically observable properties include amplitude of intraocular pressure oscillations, period of intraocular pressure oscillations, blood velocity in retrobulbar vessels, total retinal blood flow, blood velocity in retinal arteries, blood velocity in retinal capillaries, blood velocity in retinal veins, oxygen saturation in retinal arteries, oxygen saturation in retinal veins, or a combination thereof.

16. The system of claim 16, wherein the blood velocity in retrobulbar vessels includes one or more of the following: peak systolic velocity, end diastolic velocity, resistive index, full time profile along a cardiac cycle, area under the curves, presence or absence of well-defined peaks, systolic slope, and diastolic slope.

17. The system of claim 9, wherein steps a) and b) are not optional.

18. The method of claim 1, wherein steps a) and b) are not optional.
